(19) United States
(12) Patent Application Publication
(10) Pub. No.: US 2010/0211043 A1
(43) Pub. Date: Aug. 19, 2010

(54) METHODS FOR FIXING A GASTROINTESTINAL DEVICE IN THE GI TRACT

(76) Inventors: Yehiel Ziv, Tel Aviv (IL); Avinoam Nevler, Tel Aviv (IL)

Correspondence Address: David Klein;DEKEL PATENT LTD., Beit HaRof'im, 18 Menuha VeNahala Street, Room 27, Rehovot, IL

(21) Appl. No.: 12/633,872

(22) Filed: Dec. 9, 2009

Related U.S. Application Data
(60) Provisional application No. 61/152,990, filed on Feb. 17, 2009.

(51) Int. Cl. A61M 5/32

(52) U.S. Cl. 604/506; 604/187

(57) ABSTRACT

A method for fixing a gastrointestinal (GI) device in a GI tract, the method including introducing a substance into a tissue of the GI tract that reduces or eliminates the tissue's physiological capability to reject foreign bodies, and fixing a GI device into the tissue where the substance has been introduced.

### CROSS-REFERENCE TO RELATED APPLICATIONS

This application claims priority under 35 USC §119 to U.S. Provisional Patent Application, Ser. No. 61/152990, filed Feb. 17, 2009, which is incorporated herein by reference.

### FIELD OF THE INVENTION

The present invention relates generally to methods for fixing a gastrointestinal device in the GI tract.

### BACKGROUND OF THE INVENTION

PCT Patent Application PCT/IL2005/000002, to the present inventors, describes a gastrointestinal device for controlling fecal incontinence. The device includes a valve disposed in a casing which is fixed in the gastrointestinal (GI) tract (e.g., the anorectal wall) by means of fixation elements. The valve is controllable to move from a closed position, which significantly restricts passage of gastrointestinal (e.g., fecal) matter therepast, and an open position, which permits passage of gastrointestinal matter therepast. A controller is operatively connected to the valve for externally controlling the position of the valve between the closed and open positions.

The device must be properly fixed to the GI tract in order to succeed in controlling fecal incontinence. However, the tissue of the GI tract is capable of regeneration and re-growth in such a way that can cause rejection of a foreign body, such as mechanical fasteners used to secure the device in the GI tract. The problem is made worse by food traveling inside the GI tract which creates forces that tend to rip away fixation elements from the tissue. Therefore, simply trying to attach the device to the GI tract wall by suturing or by using prongs or barbs may not always succeed to hold the device permanently in place.

### SUMMARY OF THE INVENTION

The present invention seeks to provide novel methods for fixing a gastrointestinal device in the GI tract, particularly for use in controlling fecal incontinence, as is described in detail hereinbelow.

It is noted that the methods of the present invention are described hereinbelow for use with a device attached to the anorectal wall to control or treat fecal incontinence. However, the invention is not limited to this application and the invention may be used in other parts of the gastrointestinal tract as well as other lumens in the body.

### BRIEF DESCRIPTION OF THE DRAWINGS

The present invention will be understood and appreciated more fully from the following detailed description taken in conjunction with the drawing in which:

FIG. 1 is a simplified pictorial illustration of a device for injecting a substance into tissue of the GI tract, constructed and operative in accordance with an embodiment of the invention.

### DETAILED DESCRIPTION OF EMBODIMENTS

In accordance with an embodiment of the present invention, in order to enable permanent attachment of a gastrointestinal device to the tissue, especially in the GI tract, the tissue is intentionally re-modeled to change its physiological capability to reject foreign bodies by growing around them. In one embodiment of the invention this is accomplished by promoting growth of fibrotic tissue, a tough kind of tissue that does not have a foreign body response capability and can be used to anchor medical devices in place. This may be accomplished by injecting into an area of the GI tract, specifically points composing a ring around the rectum (lower part of the colon), material or fluid that causes formation of fibrotic tissue. A 5% phenol solution in almond oil has been used in the past to create fibrosis inside hemorrhoids in order to make them disappear, but it has not been suggested for use in healthy tissue. (The invention is not limited to this substance.) Injecting this substance into healthy GI tract tissue caused fibrotic “patches” to form. In one experiment, the substance was injected into points forming a ring some 8-10 cm inside the anus. This made the tissue readily accept fixation of the GI device thereto, such as by suturing or by using metal pins, hooks or barbs (for example, fixation elements described in PCT Patent Application PCT/IL2005/000002).

In accordance with an embodiment of the present invention, the GI device itself is provided with injection means in the area of fixation for injecting the substance into the tissue of the GI tract to reduce or eliminate the tissue's physiological capability to reject foreign bodies. For example, the GI device (which may be the valve device described in PCT Patent Application PCT/IL2005/000002) includes a reservoir of the substance and one or more needles through which the substance is injected into the tissue. The needle or needles may be mounted in the device and operatively connected to an actuator which pushes the needles outwards to pierce the tissue. The actuator also causes flow of the substance through the needle or needles into the tissue. The actuator may be operated or triggered by a manual device external to the body. After injection of the substance, the fixation elements (such as barbs, hooks or pins and the like) may better fix the device to the fibrotic tissue.

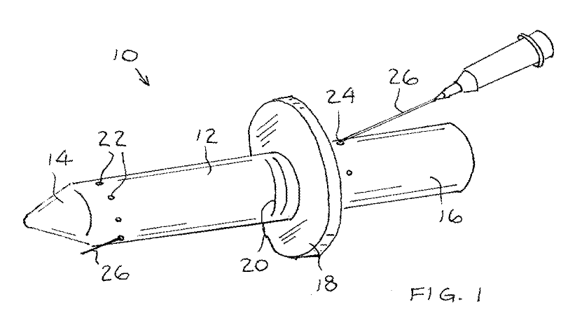

Reference is now made to FIG. 1, which illustrates an injection device 10 for injecting a substance into tissue of the GI tract, in accordance with an embodiment of the invention. Device 10 includes a slender hollow body 12 with a tapered distal end 14 and a handle 16 at the proximal end. A hand guard 18, such as a disc, is assembled on body 12 such as by screwing on threads 20 formed on body 12. A plurality of distal apertures 22 are formed on body 12 proximal to distal end 14, and may be either equally spaced around the perimeter of body 12 or unequally spaced therearound. One or more proximal apertures 24 are formed on the proximal side of hand guard 18. A long needle 26 may be introduced through any of the proximal apertures 24 so that the tip of the needle exits one of the distal apertures 22. In this way the needle 26 may pierce the tissue for introducing a substance into the tissue, such as for making the tissue fibrotic as above.

Device 10 may be used for creating a fibrotic ring of tissue in the region of the rectum for any treatment purposes and not just for fixation of a GI device. For example, in accordance with an embodiment of the invention, the created growth of fibrotic tissue is used to narrow the rectal canal, thereby reducing the wall tension and allowing easier fixation. Alternatively, the created growth of fibrotic tissue can be used as a method for treatment of low grade incontinence.

The scope of the present invention includes both combinations and subcombinations of the features described hereinabove as well as modifications and variations thereof which would occur to a person of skill in the art upon reading the foregoing description and which are not in the prior art.

What is claimed is:

1. A method for fixing a gastrointestinal (GI) device in a GI tract, the method comprising:
introducing a substance into a tissue of the GI tract that reduces or eliminates the tissue's physiological capability to reject foreign bodies; and
fixing a GI device into the tissue where the substance has been introduced.

2. The method according to claim 1, wherein introducing the substance into the tissue of the GI tract comprises injecting the substance at discrete points forming a ring, the substance causing fibrotic patches to form in the tissue.

3. Apparatus for fixing a gastrointestinal (GI) device in a GI tract comprising:
an injection device for injecting a substance into tissue of the GI tract, said injection device comprising a slender hollow body with a tapered distal end and a handle at a proximal end thereof, a plurality of distal apertures being formed on said body proximal to said distal end;
a hand guard assembled on said body, one or more proximal apertures being formed on a proximal side of said hand guard; and
a needle insertable through said one or more proximal apertures so that a tip of said needle exits one of said distal apertures.

4. The apparatus according to claim 3, wherein distal apertures are equally spaced around a perimeter of said body.

5. The apparatus according to claim 3, wherein distal apertures are unequally spaced around a perimeter of said body.

6. A method for treating a GI tract comprising:
introducing a substance into a rectal canal that creates fibrotic tissue in the rectal canal so as to narrow the rectal canal and reduce wall tension therein.
